(19) United States
(12) Patent Application Publication
(10) Pub. No.: US 2019/0151371 A1
(43) Pub. Date: May 23, 2019

(54) PROCESS FOR CONTINUOUS CELL CULTURE OF ISLET CELLS

(71) Applicant: Georgetown University, Washington, DC (US)

(72) Inventors: Ian Gallicano, Alexandria, VA (US); Samiksha Mahapatra, Washington, DC (US)

(73) Assignee: Georgetown University, Washington, DC (US)

(21) Appl. No.: 16/308,582

(22) PCT Filed: Jun. 9, 2017
(86) PCT No.: PCT/US2017/036670

§ 371 Date: Dec. 10, 2018

Related U.S. Application Data
(60) Provisional application No. 62/347,767, filed on Jun. 9, 2016.

(51) Int. Cl. A61K 35/39; A61P 1/18; C12N 5/00; C12N 5/09; G01N 33/574; C12Q 1/04

(52) U.S. Cl. CPC A61K 35/39; A61P 1/18; C12Q 1/045; C12N 5/0693; G01N 33/57438; C12N 5/0018

(57) ABSTRACT

The present invention is directed towards methods of culturing pancreatic islet cells, with the methods comprising culturing pancreatic islet cells in the presence a cell culture medium while inhibiting the activity of Rho kinase (ROCK) in the cells during culturing.

### BACKGROUND OF THE INVENTION

### Field of the Invention

The present invention is directed towards methods of culturing pancreatic islet cell, with the methods comprising culturing the cells in a cell culture medium while inhibiting the activity of Rho kinase (ROCK) in the cells during culture. The present invention is also directed towards methods of using these continuously cultured islet cells.

### Background of the Invention

While islet cell transplantation is an effective treatment for type-I diabetes, this method suffers from myriad drawbacks. One of the primary limitations of current transplantation procedures is the necessity of harvesting islet cells from more than one donor. For example, using multiple donors decreases availability of tissue for transplant. In addition, transplanting cells from more than one donor increases the chances of graft rejection in recipients.

To date, isolating post-natal pancreatic or islet cell progenitor cells has been difficult or not feasible. Indeed, it still not entirely clear where any such progenitor cells, if they exist, reside in the pancreas. Moreover, procedures designed to isolate progenitor cells, such as surgical techniques, have been associated with pancreatic inflammation and cell apoptosis. Even if it were feasible to isolate islet progenitor cells, it is highly likely that the isolation procedures would damage or even destroy the cells to the point of there being insufficient numbers for in vitro expansion.

Clearly, methods of expanding mature islet cells in vitro are needed. Such methods of expanding islet cell populations would reduce graft rejection, increase tissue supplies and avoid problems associated with isolating islet progenitor cells.

### SUMMARY OF THE INVENTION

The present invention is directed towards methods of culturing pancreatic islet cell, with the methods comprising culturing the cells in a cell culture medium while inhibiting the activity of Rho kinase (ROCK) in the cells during culture. The present invention is also directed towards methods of using these continuously cultured islet cells.

The present invention is also directed towards methods of producing conditionally immortalized pancreatic islet cells, with the methods comprising culturing the cells in the presence of a cell culture medium while inhibiting the activity of ROCK in the cells. Culturing the islet cells in such conditions will produce conditionally immortalized pancreatic islet cells.

The present invention is also directed towards methods of producing at least partially differentiated pancreatic islet cells comprising culturing for a set time pancreatic islet cells in the presence of a cell culture medium while inhibiting the activity of ROCK in the cells to produce conditionally immortalizing pancreatic islet cells. After culturing the conditionally immortalized pancreatic islet cells in these conditions, the conditionally immortalized pancreatic islet cells are placed in conditions that promote differentiation of the conditionally immortalized pancreatic islet cells.

The present invention is also directed towards methods of stimulating growth of pancreatic islet cells, with the methods comprising culturing the pancreatic islet cells in the presence of a cell culture medium while inhibiting the activity of ROCK in the cells. Culturing the pancreatic islet cells in such conditions will stimulate pancreatic islet cells to grow, whereas otherwise the cells may not grow.

### BRIEF DESCRIPTION OF THE DRAWINGS

FIG. 1 depicts the structures of select ROCK inhibitors.

FIG. 2 depicts a cluster gram of gene expression in islet cells isolated from four different patients. The cluster gram shows gene expression in control cells, late-passaged islet cells using the methods of the present invention in expansion medium (“ICE Medium”) and re-derived islet cells (Re-De Islets). Re-derived islets are small tissues containing endocrine producing cells similar to primary islets of Langerhans found in the pancreas. Re-derived islets were generated using a two-step process beginning with isolated and purified pancreatic, primary Islets of Langerhans. Primary islets were first cultured in ICE medium, a DMEM-based medium that is supplemented with a Rho kinase inhibitor and 10% human serum. This medium was used to reactivate the cell cycle within primary islets. Once activated, islets can be passaged and expanded from one dish to many dishes. Once expanded, the second step for re-deriving islets was to culture them in a different CMRL medium that was supplemented with growth factors that induce or reactivate the Islet differentiation pathway. After 14 days in the re-derivation medium, the tissue contained all five cells types found in a primary islet. These new sets of tissue are referred to herein as re-derived islets.

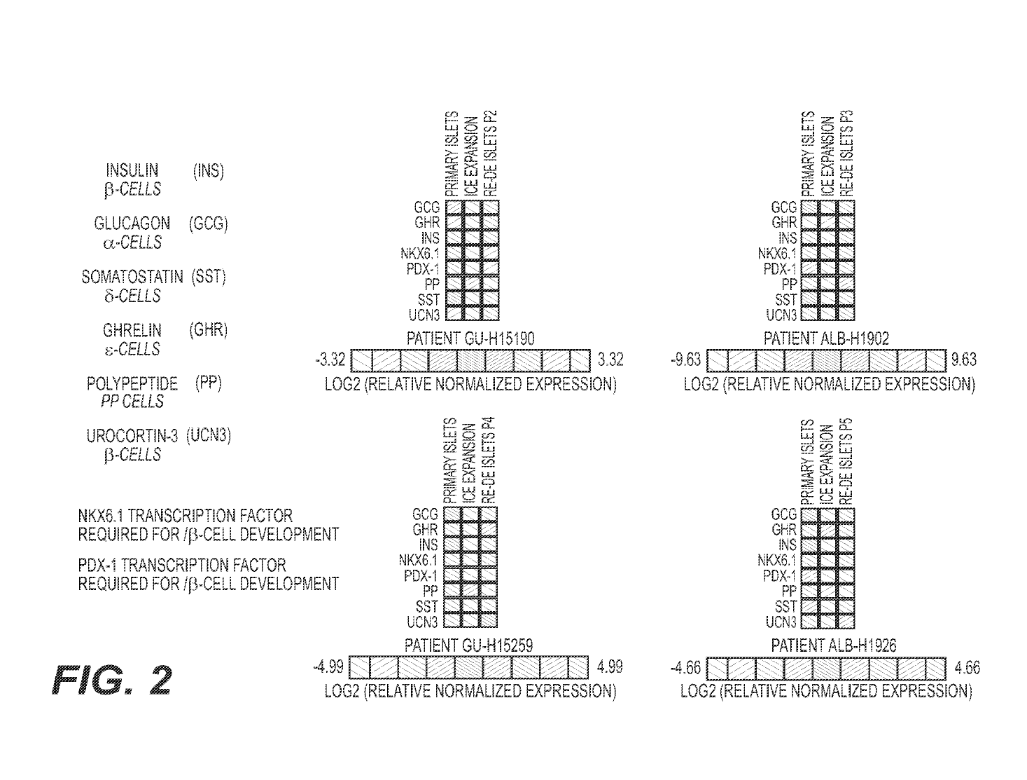

FIG. 3 depicts stained, isolated primary islets using a 30-second stain in dithizone (DTZ), followed by culture in the culture conditions described herein (ICE Medium). 3A shows islets shortly after isolation, using a pipetteman from non-stained aciner tissue, and staining positively for DTZ. 3B shows the same cells after nine days in the culture conditions described herein (ROCK inhibition and human serum). The islets re-enter the cell cycle, enabling indefinite passaging. Arrows point to small clusters of cells. Few cells, however, stain for DTZ. 3C shows the cells 14 days in standard CMRL medium used for islet cell culture, plus T3 and Alk5i inhibitor, used for islet cell culture. The islet clusters stain positive for DTZ (arrows). Scale bar in A=100 μm for A and 50 μm for B and C.

FIG. 4 depicts confocal microscopy of conditionally immortalized islet cells showing the same markers as primary human islets. 4A-4E show primary islet cells that were stained for protein markers that are “typical” of islet cells: insulin, glucagon, C-peptide, somatostatin, gherelin, and polypeptide (PP). Antibodies used are labeled in each figure. Islets were placed into the culture conditions described herein (ICE Medium) for at least 9-10 days, passaged once, and then re-cultured in CMRL media supplemented with 10% human serum along with 10 μM Alk5ii inhibitor and 1 μM T3 for 14-21 days. 4F-4J show that the cells still stain positive for the proteins assayed when initially cultured, prior to expansion. The green staining C-peptide and the magenta staining insulin virtually overlap as shown by the white staining (green+magenta=white) merged image. 4K-4L show that transcription factor markers specific to islet cells (Mafa and Nkx6.1) were clearly present in some of the conditionally immortalized islet cells. Scale bar in A=150 μm for 4A-4E. Scale bar in 4F=20 μm for 4 F-4F″ and 75 μm in 4G-4J. Scale bar in 4K=20 μm in 4K-4K″ while scale bar in 4L=7.5 μm for 4 L-4L″.

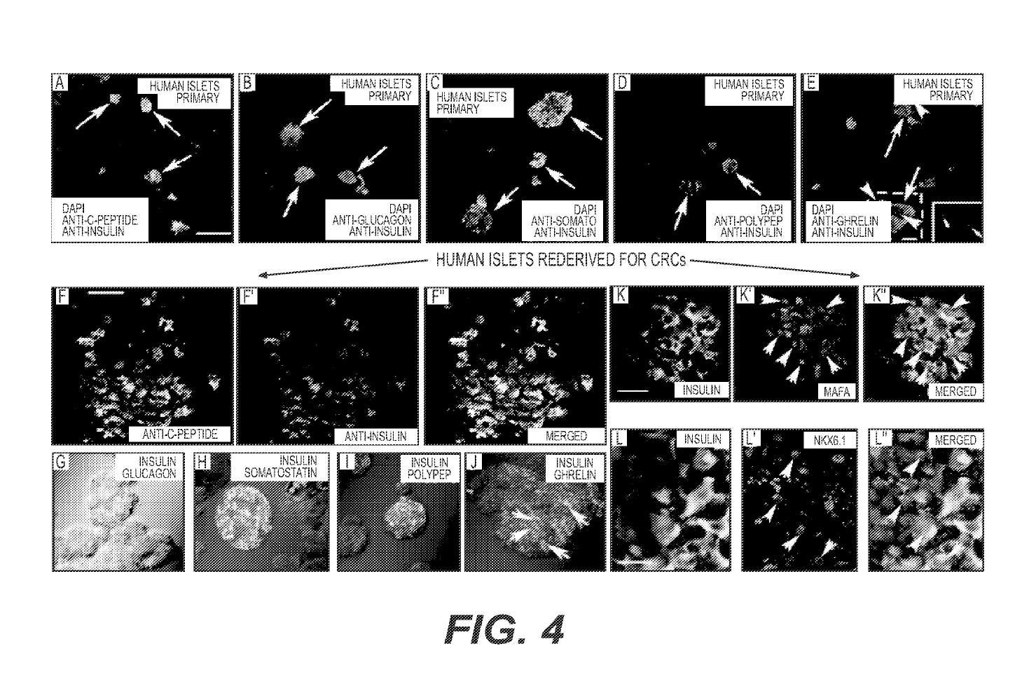

FIG. 5 depicts rt-PCR of expressed genes known to be important for islet function in control human islets (Top Panel). After 10 passages in using the methods of the present invention, expression of some genes, e.g., Isl1, insulin, ghrelin, falls at or below the level of detection (middle panel). 14-21 days after removal of Y-27632 and culture in re-derivation medium, the islet genes are once again expressed.

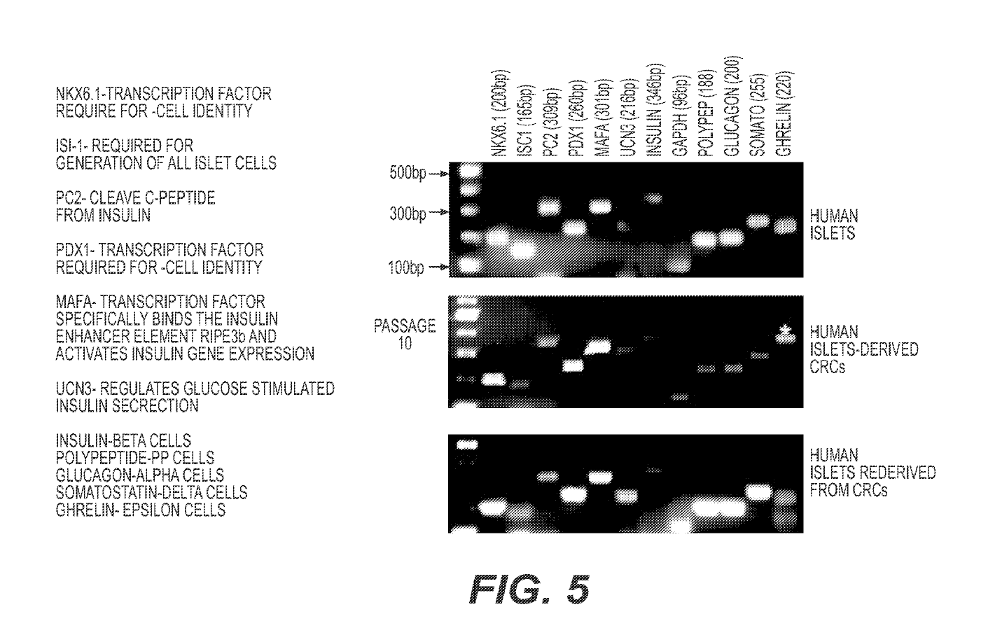

FIG. 6 depicts ELISA analyses of C-peptide expression in response to varying levels of glucose in primary islets (Blue large Diamonds) and conditionally immortalized islets that were re-derived and placed in CMRL medium (Red Diamonds). The measured levels were placed on the standard curve included with the ELISA kit. Virtually no differences in glucose responses are observed between primary and re-derived islets at both 1 mM and 10 mM glucose.

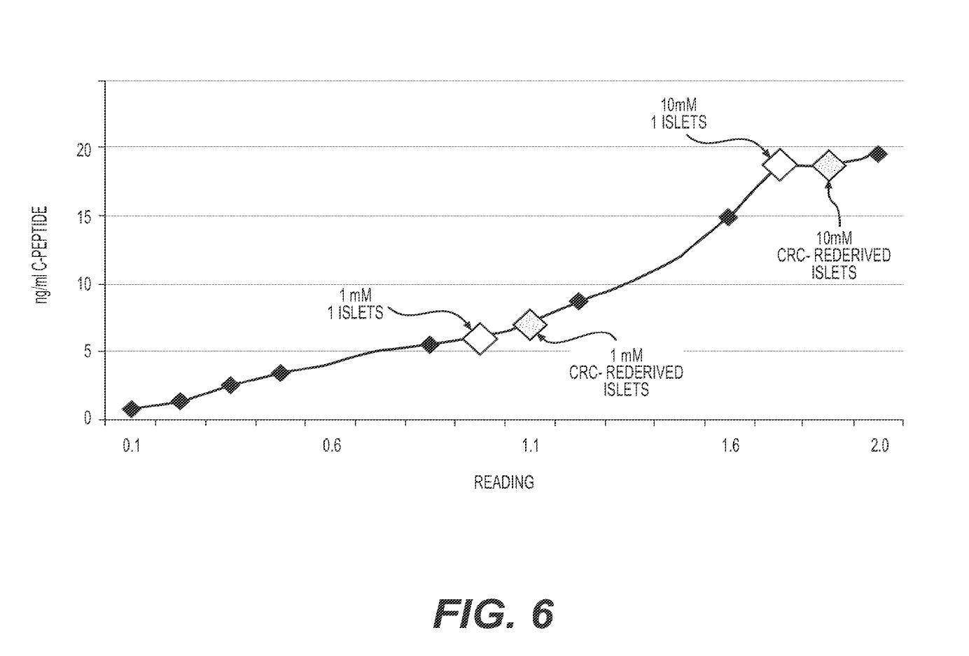

FIG. 7 depicts human islet cells re-derived from conditionally immortalized islet cells behave similarly as a normal human islet freshly isolated from the pancreas with respect to calcium flux in response to high glucose. Lower left panel shows calcium flux in individual cells in response to 10 mM glucose. Right graph shows the average signal from all cells at low and high glucose levels.

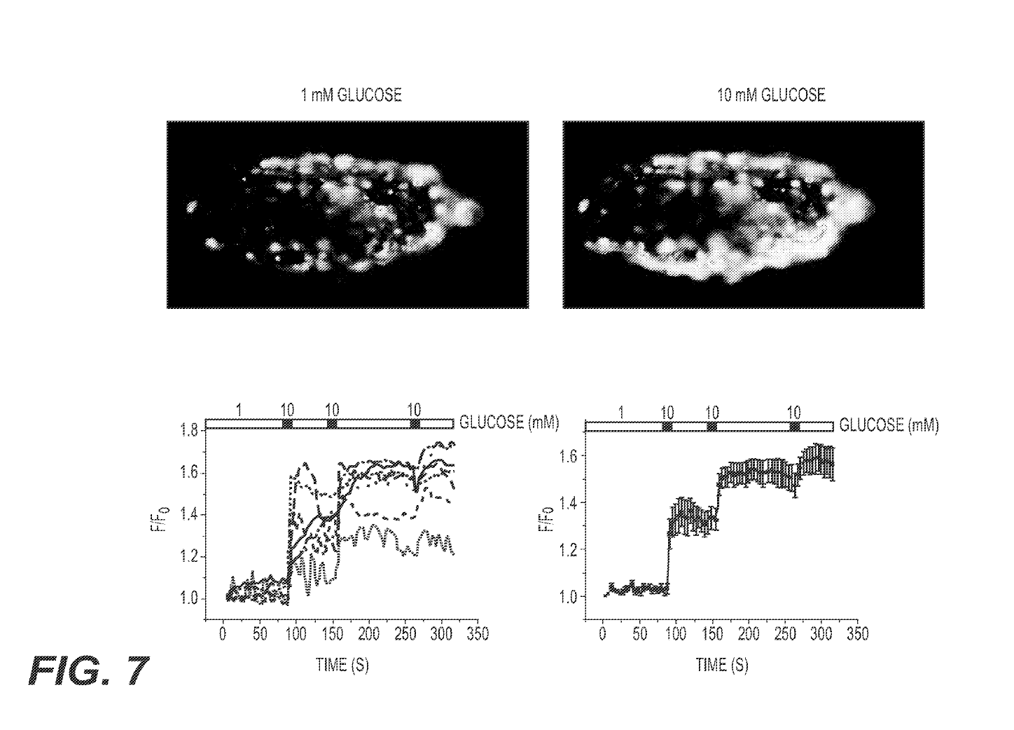

FIG. 8 depicts a Western Blot of islets using an antibody directed against PhospoTyr14/Thr15 CDK2 showing that CDK2 in primary islets is phosphorylated (Lane 2); however, over 7-10 days in the culture conditions described herein (ICE Medium), this phosphorylation dissipates as the islets become expandable (lanes 3-5). After removal of ROCK inhibition, CDK2 becomes phosphorylated again on Thr/Tyr15 (Lanes 6-8).

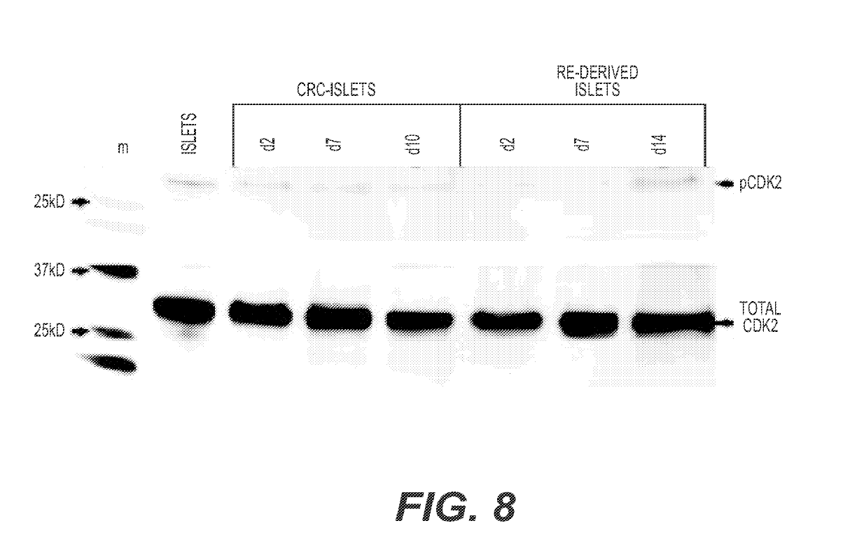

FIG. 9 depicts results obtained from a glucose challenge in primary islet cells and re-derived (RD) cells that were generated using the methods of the claimed invention,

### DETAILED DESCRIPTION OF THE INVENTION

The present invention is directed towards methods of culturing pancreatic islet cell, with the methods comprising culturing the cells in a cell culture medium while inhibiting the activity of Rho kinase (ROCK) in the cells during culture. The present invention is also directed towards methods of using these continuously cultured islet cells.

As used herein, the term “islet cell” or “pancreatic islet cell” refers to a cell or cells that are typically found within the Islets of Langerhans in a functioning pancreas. Any cell normally found within the Islets of Langerhans is considered a “islet cell” or a pancreatic islet cell” for the purposes of the present invention. In one embodiment, an islet cell is a “beta cell” or a “beta islet cell,” which normally produces insulin. Other cells within the islets of Langerhans include “alpha cells” or “alpha islet cells,” which normally produce glucagon, “delta cells” or “delta islet cells,” which normally produce somatostatin, “epsilon cells” or “epsilon islet cells,” which normally produce ghrelin, “gamma cells” or “gamma islet cells,” (or “PP cells) which normally produce pancreatic polypeptide (PP). Any of these cells are considered to be “islet cells” for the present invention. Moreover, the islet cells used in the methods of the present invention can be a mixture of one or more cell types (alpha, beta, gamma, delta and/or epsilon cells) or the islet cells used in the methods of the present invention can be a pure or substantially pure population of alpha, beta, gamma, delta and/or epsilon cells.

The cells can be from any animal, including but not limited to any mammal, such as mouse, rat, canine, feline, bovine, equine, porcine, non-human and human primates. Mammalian cells particularly suitable for cultivation in the present media include islet cells of human origin, which may be primary cells derived from a pancreas. In addition, transformed cells or established cell lines islet cell lines can also be used. The cells used in the present invention may be normal, healthy cells that are not diseased or not genetically altered, or the cells may be diseased or genetically altered. Accordingly, “diseased islet cells” are a subset of islet cells herein. “Diseased cells” means that the islet cells are from an abnormal pancreas, such as from a neoplasia, a hyperplasia or malignant tumor or benign tumor including of an animal. In one embodiment, the cells are primary or secondary human islet cells from a sample of normal or abnormal tissue. In another embodiment, the cells are not primary cells, such as cells from an established cell line, transformed cells, thawed cells from a previously frozen collection and the like. Animal cells for culturing by the present invention may be obtained commercially, for example from ATCC (Rockville, Md.), Cell Systems, Inc. (Kirkland, Wash.), Clonetics Corporation (San Diego, Calif.), BioWhittaker (Walkersville, Md.) or Cascade Biologicals (Portland, Oreg.).

As used herein, primary islet cells are cells that have been taken directly from living tissue, such as a biopsy, and have not been passaged or only passaged one time. Thus, primary cells have been freshly isolated, often through tissue digestion and plated. Provided the cells have been passaged one time or less, primary cells may or may not be frozen and then thawed at a later time. In addition, the tissue from which the primary islet cells are isolated may or may not have been frozen of preserved in some other manner immediately prior to processing.

The islet cells for use the present invention are not undifferentiated, embryonic stem cells. Thus, the phrase islet cell as used herein automatically excludes undifferentiated embryonic stem cells. As used herein and in the art, embryonic stem cells are undifferentiated cells that have the capacity to regenerate or self-renew indefinitely. The islet cells used in the methods herein may or may not be adult stem cells, i.e., progenitor cells. As used herein, adult stem cells are isolated from tissues of an animal and are less differentiated than completely differentiated cells, but are more differentiated than embryonic stem cells. In one embodiment, the islet cells cultured according to the methods of the present invention are adult islet stem cells, i.e., islet progenitor cells. In another embodiment of the present invention the islet cells cultured according to the methods of the present invention are not adult islet stem cells, i.e., not islet progenitor cells. The islet cells used in the present invention, even if considered to be islet progenitor cells, would not normally have the capacity for indefinite self-renewal. Moreover, the islet cells are not completely undifferentiated cells upon initial isolation and plating in that the cells may possess cell surface markers not typically associated with undifferentiated stem cells, or conversely the islet cells do not possess cell surface all markers typically associated with undifferentiated stem cells.

When isolating primary cells, tissue should ideally be handled using standard sterile techniques and a laminar flow safety cabinet. In one embodiment, a single needle biopsy is sufficient to isolate enough primary cells to begin the cell culture methods of the present invention. In the case of a tissue biopsy, tissue can be cut into small pieces using sterile instruments. The small pieces can then be washed several times with sterile saline solution or other buffer, such as PBS, that may or may not be supplemented with antibiotics or other ingredients. After washing, the pieces are often, but need not be, treated with an enzymatic solution such as, but not limited to collagenase, dispase or trypsin, to promote dissociation of cells from the tissue matrix.

Dispase is often used to dissociate epithelium, including pancreatic tissue, from the underlying tissue. This intact epithelium may then be treated with trypsin or collagenase. These digestion steps often results in a slurry containing dissociated cells and tissue matrix. The slurry can then be centrifuged with sufficient force to separate the cells from the remainder of the slurry. The cell pellet can then be removed and washed with buffer and/or saline and/or cell culture medium. The centrifuging and washing can be repeated any number of times. After the final washing, the cells can then be washed with any suitable cell culture medium. Of course, the digestion and washing steps need not be performed if the cells are sufficiently separated from the underlying tissue upon isolation, such as the case in a needle biopsy or if isolated from the circulation. Cells may or may not be counted using an electronic cell counter, such as a Coulter Counter, or they can be counted manually using a hemocytometer. Of course, the cells need not be counted at all.

For the purposes of the present invention cells are no longer considered to be primary cells after the cells have been passaged more than once. In addition, cells passaged once or more and immediately frozen after passaging are also considered not to be primary cells when thawed. In select embodiments of the present invention, the islet cells are initially primary cells and, through the use of the methods of the present invention, become non-primary cells after passaging.

By “cell culture” or “culture” is meant the maintenance of the cells in an artificial, in vitro environment. The term “cell culture” also encompasses cultivating individual cells and tissues.

The cells being cultured according to the present invention, whether primary or not, can be cultured and plated or suspended according to the experimental conditions as needed by the technician. The examples herein demonstrate at least one functional set of culture conditions that can be used in conjunction with the methods described herein. If not known, plating or suspension and culture conditions for a given animal cell type can be determined by one of ordinary skill in the art using only routine experimentation. Cells may or may not be plated onto the surface of culture vessels, and, if plated, attachment factors can be used to plate the cells onto the surface of culture vessels. If attachment factors are used, the culture vessels can be precoated with a natural, recombinant or synthetic attachment factor or factors or peptide fragments thereof, such as but not limited to collagen, fibronectin and natural or synthetic fragments thereof.

The cell seeding densities for each experimental condition can be manipulated for the specific culture conditions needed. For routine culture in plastic culture vessels, an initial seeding density of from about 1×10⁴ to about 1×10⁷ cells per cm² is fairly typical, e.g., 1×10⁶ cells are often cultured in a 35 mm²-100 mm² tissue culture petri dish. Using the methods of the present invention, however, even a single cell can be plated or suspended initially. Thus, the methods of the present invention can be performed using 1, 2, 3, 4, 5, 6, 7, 8, 9, 10, 20, 30, 40, 50, 60, 70, 80, 90, 100 or more cells for an initial cell seeding. Of course, higher cell seeding numbers can be used, such as but not limited to 1×10³, 1×10⁴, 1×10⁵ and so on. Cell density can be altered as needed at any passage.

Mammalian cells are typically cultivated in a cell incubator at about 37° C. at normal atmospheric pressure. The incubator atmosphere is normally humidified and often contain about from about 3-10% carbon dioxide in air. Temperature, pressure and CO₂ concentration can be altered as necessary, provided the cells are still viable. Culture medium pH can be in the range of about 7.1 to about 7.6, in particular from about 7.1 to about 7.4, and even more particular from about 7.1 to about 7.3.

Cell culture medium is normally replaced every 1-2 days or more or less frequently as required by the specific cell type. As the islet cells approach confluence in the culture vessel, they would normally be passaged. As used herein a cell passage is a term that is used as it is in the art and means splitting or dividing the cells and transferring a portion of the cells into a new culture vessel or culture environment. Most likely, the islet cells used in the methods of the present invention will be adherent to the cell culture surface and will need to be detached. Methods of detaching adherent cells from the surface of culture vessels are well-known and commonly employed and can include the use of enzymes such as trypsin.

A single passage refers to when a technician splits or manually divides the cells one time and transfers a smaller number of cells into a new vessel or environment. When passaging, the cells can be split into any ratio that allows the cells to attach and grow. Thus, at a single passage the cells can be split in a 1:2 ratio, 1:3, 1:4, 1:5 etc. Passaging cells, therefore, is not necessarily equivalent to population doubling. As used herein a population doubling is when the cells divide in culture one time such that the number of cells in culture is approximately doubled. Cells need to be counted to determine if a population of cells has doubled, tripled or multiplied by some other factor. In other words, passaging the cells and splitting them in a 1:3 ratio for further culturing in vitro is not to be taken as the equivalent that the cell population has tripled.

In one embodiment of the present invention, the islet cells are continuously cultured in vitro. As used herein, “continuous culturing” is the notion that the cells continually divide and reach or approach confluence or a certain density in the cell culture vessel such that the cells require passaging and fresh medium to maintain their health. Thus, the concept of “continuously culturing” is similar to the concept that the islet cells would be “immortalized.” Accordingly, the term “conditionally immortalized” refers to the ability of the cells to divide in the prescribed culture conditions indefinitely, i.e., regardless of the number of passages, such that the islet cells growing in the prescribed conditions would need to be passaged to maintain their health. In one embodiment, when cultured using the present methods and conditions of the present invention, normal islet cells can continue to grow and divide for at least 5, 10, 15, 20, 25, 30, 35, 40, 45, 50, 55, 60, 65, 70, 75, 80, 85, 90, 95, 100, 125, 150, 175, 200, 250 or 300 passages or more.

The present invention is also directed towards methods of stimulating growth of islet cells, in particular normal islet cells, in vitro with the methods comprising culturing the islet cells in the presence of a cell culture medium while inhibiting the activity of ROCK in the islet cells. Culturing the islet cells in such conditions will stimulate the islet cells to grow or proliferate, whereas otherwise the islet cells may not grow. In one specific embodiment, the cells can grow on plates or in suspension in tight clusters, i.e., the cells become tightly adherent. In another embodiment, the cells grow in suspension and may or may not grow in clusters. In one embodiment, the cultured islet cells form junctions involving e-cadherin, non-muscle myosin, p120 catenin and gap junction protein such as but not limited to connexin 36. These types of junctions can be assayed according to Li, D. et al., J. Cell Biol., 191(3):631-644 (2010), which is incorporated by reference.

As used herein and throughout the specification, “cell growth” refers to cell division, such that one “mother cell” divides into two “daughter cells.” As used herein, “cell growth” does not refer to an increase in the actual size of the cells. Stimulation of cell growth can be assayed by plotting cell populations over time. A cell population with a steeper growth curve can said to be growing faster than a cell population with a curve not as steep. Growth curves can be compared for various treatments between the same cell types, or growth curves can be compared for different cell types, e.g., abnormal versus normal islet cells, with the same conditions.

The late passage islet cells, in particular late passage normal islet cells, of the present invention may or may not be characterized by their telomere length. As normally happens, the length of the telomeres generally shortens as cells divide. A cell will normally stop dividing when the average length of telomeres is reduced to a critical length, e.g., 4 kb. In the present invention, the average telomere length of late passage cells may be reduced to a length of as little as 2 kb and continue to grow. The average telomere length is readily determined using routine methods and techniques in the art. Thus in one embodiment, the present invention provides islet cells, in particular normal islet cells, capable of dividing in the culture conditions of the present invention, wherein the average telomere length of the islet cells is shorter than the average telomere length of islet cells that would normally not divide when placed under different or heretofore routine culture conditions. Thus, the methods of the present invention are capable of generating conditionally immortalized islet cells, in particular normal conditionally immortalized islet cells, whereby the cells have an average telomere length that is less than the average telomere length of islet cells that are normally capable of dividing and whereby the conditionally immortalized islet cells are still capable of dividing in spite of their reduced telomere length. To be clear, islet cells, in particular normal islet cells will typically stop dividing when the average telomere length is reduced to a certain length even when placed in culture conditions currently considered in the art to be acceptable or even optimal for culturing islet cells.

Such currently acceptable or optimal conditions for culturing epithelial cells, including islet cells, generally include culturing cells in well-defined, or synthetic, serum-free medium. For example, culturing islet cells normally involves culturing in islet cell-specific medium, with added serum. Thus, “currently acceptable” or “currently optimal” culture conditions are culture conditions where the medium includes serum, such as but not limited to human serum at about 10%. “Currently acceptable” or “currently optimal” culture conditions may also include the use of synthetic or well-defined medium, for example the use of islet-specific cell medium for islet cells. Thus the methods of the present invention provide the unexpected results of being able to culture and passage islet cells, in particular normal islet cells, long after one would have been able to do so using currently acceptable or currently optimal conditions.

As used herein, the term “conditionally immortalized” indicates that the islet cells have a reduced average telomere length over the average telomere length of normally senescent islet cells yet are still capable of unlimited growth in the prescribed conditions. When determining if a cell is conditionally immortalized, it may be necessary to compare the average telomere length of the conditionally immortalized cells with the average telomere length of non-conditionally immortalized islet cells that would normally be senescent in vitro. The phrase “normally senescent” is used to mean a population of islet cells that, but for being cultured in the conditions outlined herein, would a reduced capacity of dividing further in vitro and thus would not need to be passaged any further. Therefore, the invention provides methods of conditionally immortalizing islet cells, in particular normal islet cells, comprising culturing the islet cells in the presence of a cell culture medium while inhibiting the activity of Rho kinase (ROCK) in the islet cells during culturing.

As used herein, “conditionally immortalized cells” are not induced pluripotent stem cells (IPS Cells). Induced pluripotent stem cells are cells that have been re-programmed to resemble and function like pluripotent stem cells such that the IPS cells are capable of generating a plurality of different tissues. In contrast, the conditionally immortalized islet cells of the present invention may become less differentiated than terminally differentiated islet cells but are able to proliferate under the conditions outlined herein. By “less differentiated” this term is used to mean that the cells, while in the inventive culture conditions described herein may not express the full complement of markers that fully differentiated islet cells normally express. In the alternative, “less differentiated” can also mean that the cells, while in the inventive culture conditions described herein may take on a less developed phenotype than that that of fully differentiated islet cells. As defined herein, conditionally immortalized islet cells of the present invention do not acquire the ability to differentiate into multiple tissue types. In one embodiment of the present invention, the conditionally immortalized islet cells generated by the methods described herein retain the ability to differentiate back into fully differentiated islet cells. In another embodiment of the present invention, the conditionally immortalized islet cells generated by the methods described herein retain the ability to differentiate back into fully differentiated islet cells, but no other cell type. In another embodiment, the conditionally immortalized islet cells generated by the methods described herein do not retain the ability to fully differentiate back into fully differentiated islet cells.

The islet cells can grow, become in need of continuous culturing and/or become conditionally immortalized in vitro without apparent change to the karyotype of the cells after any number of passages. Accordingly, the methods of the present invention comprise continuously culturing islet cells whereby the cells' karyotype at any passage is not altered or is not substantially altered when compared to the karyotype of primary islet cells or early passage islet cells. An alteration of a cell's karyotype includes but is not limited to duplication or deletion of chromosomes or portions thereof and/or translocation of a portion of one chromosome to another. Identifying a karyotype and alterations thereof are common techniques in the art. Accordingly, one embodiment of the present invention is directed to late passage islet cells, in particular late passage normal islet cells wherein the late passage islet cells have (a) an unaltered karyotype when compared to the karyotype of primary islet cells or early passage islet cells or (b) an unaltered karyotype when compared to the karyotype of initially thawed islet cells. As used herein, a late passage islet cell is defined as an islet cell that has gone through at least 5, 10, 15, 20, 25, 30, 35, 40, 45, 50, 55, 60, 65, 70, 75, 80, 85, 90, 95, 100, 125, 150, 175, 200, 250 or 300 passages or more.

The present invention is also directed to conditionally immortalized islet cells, in particular conditionally immortalized normal islet cells. In select embodiments, the conditionally immortalized islet cells, in particular the conditionally immortalized normal islet cells have (a) an unaltered karyotype when compared to the karyotype of primary islet cells or early passage islet cells or (b) an unaltered karyotype when compared to the karyotype of initially thawed islet cells.

In select embodiments, the methods of the present invention do not use feeder cells. The term “feeder cells” is used herein as it is in the art. Namely, feeder cells are cells that are co-cultured with the “target cells” and share the same medium and vessel as the target cells. The term “feeder cells” is well-known in the art.

In another embodiment, the methods also do not use medium conditioned with feeder cells, i.e., the methods do not use “conditioned medium.” The term conditioned medium is well-known in the art.

The present invention also relates to novel compositions. The novel compositions can be useful for culturing islet cells.

The cell culture media of the present invention can be any aqueous-based medium and can include any “classic” media such as, but not limited to Dulbecco's Modified Eagle Medium (DMEM) and/or F12 medium. Other cell culture media used in the methods of the present invention include but is not limited to Connaught Medical Research Laboratories (CMRL) 1066 medium (500 ml) supplemented with L-glutamine (5 ml) and 1% Penicillin/Streptomycin (5 ml), 10% human serum (50 ml). The culture medium can also be combinations of any of the classical medium, such as but not limited to CMRL 1066 with and without supplements.

Additional ingredients may be added to the culture medium used in the methods of the present invention. Such additional ingredients include but are not limited to, amino acids, vitamins, inorganic salts, adenine, ethanolamine, D-glucose, heparin, N-[2-hydroxyethyl]piperazine-N′-[2-ethanesulfonic acid] (HEPES), hydrocortisone, insulin, lipoic acid, phenol red, phosphoethanolamine, putrescine, sodium pyruvate, triiodothyronine (T3), thymidine, transferrin and Alk5ii inhibitor. Alternatively, insulin and transferrin may be replaced by ferric citrate or ferrous sulfate chelates. Each of these additional ingredients is commercially available.

Amino acid ingredients which may be included in the media of the present invention include but are not limited to, L-alanine, L-arginine, L-asparagine, L-aspartic acid, L-cysteine, L-glutamic acid, L-glutamine, glycine, L-histidine, L-isoleucine, L-leucine, L-lysine, L-methionine, L-phenylalanine, L-proline, L-serine, L-threonine, L-tryptophan, L-tyrosine and L-valine.

Vitamin that may be added include but are not limited to biotin, choline chloride, D-Ca⁺²-pantothenate, folic acid, i-inositol, niacinamide, pyridoxine, riboflavin, thiamine and vitamin B12.

Inorganic salt ingredients which may be added include but are not limited to calcium salt (e.g., CaCl₂), CuSO₄, FeSO₄, KCl, a magnesium salt, e.g., MgCl₂, a manganese salt, e.g., MnCl₂, sodium acetate, NaCl, NaHCO₃, Na₂HPO₄, Na₂SO₄ and ions of the trace elements selenium, silicon, molybdenum, vanadium, nickel, tin and zinc. These trace elements may be provided in a variety of forms, preferably in the form of salts such as Na₂SeO₃, Na₂ SiO₃, (NH₄)₆Mo₇ O₂₄, NH₄ VO₃, NiSO₄, SnCl and ZnSO.

Additional ingredients include but are not limited to heparin, epidermal growth factor (EGF), at least one agent increasing intracellular cyclic adenosine monophosphate (cAMP) levels, and at least one fibroblast growth factor (FGF). Heparin, EGF, the cAMP-increasing agent(s) and FGF(s) may be added to the basal medium or they may be admixed in a solution of, for example, Dulbecco's Phosphate Buffered Saline (DPBS) and stored frozen until being added to basal medium to formulate the medium to be used in the methods of the present invention.

Heparin may be obtained commercially. Heparin is added to the present media primarily to stabilize the activity of the growth factor components, for example FGF. If heparin is used, it may be added to the basal medium at a concentration of about 1-500 U.S.P. units/liter. EGF is available commercially. If EGF is used, it may be added to the basal medium at a concentration of about 0.00001-10 mg/L.

A variety of agents that increase intracellular cAMP levels may be used in formulating the media of the present invention. Included are agents which induce a direct increase in intracellular cAMP levels, e.g., dibutyryl cAMP, agents which cause an increase in intracellular cAMP levels by an interaction with a cellular G-protein, e.g., cholera toxin and forskolin, agents which cause an increase in intracellular cAMP levels by acting as agonists of β-adrenergic receptors, e.g., isoproterenol, and agents which cause an increase in intracellular cAMP levels by inhibiting the activities of cAMP phosphodiesterases, e.g., isobutylmethylxanthine (IBMX) and theophylline. These cAMP-increasing agents are available commercially.

The culture medium used in the methods of the present invention comprises serum or a serum replacement. The serum can be in a concentration (v/v) of from about 1% to about 35%. In select embodiments, the serum is at a concentration of from about 1% to about 20%, or from about 1% to about 15%, or from about 1% to about 10%, or from about 1% to about 5%. If a serum substitute or serum replacement is used, these can be added to the medium according to the manufacturer's suggested protocol. Examples of serum substitutes include but are not limited to commercially available substitutes such as Ultroser™ from Pall Corporation, milk or milk fractions such as but not limited to nonfat dry milk filtrate.

In specific embodiments, the serum used in the methods of the present invention is not bovine or calf serum. In more specific embodiments, the serum used in the methods of the present invention is serum from a primate. In even more specific embodiments, the serum used in the methods of the present invention is human serum.

The range of Ca⁺² concentration used in the embodiments of the present invention can vary. In one embodiment, the concentration of Ca⁺² in the medium used in the methods of the present invention is from 0.1 mM to 10.0 mM. In more specific embodiments, the concentration of Ca⁺² in the medium used in the methods of the present invention can be from about 0.2 mM to about 8 mM, from about 0.4 mM to about 7 mM, from about 0.5 mM to about 5 mM, from about 0.8 mM to about 4 mM, from about 1.0 mM to about 3 mM, from about 1.2 mM to about 2.8 mM, from about 1.4 mM to about 2.6 mM and from about 1.5 mM to about 2.5 mM.

The methods of the present invention comprise inhibiting rho associated coiled-coil protein kinase (ROCK) in the culture. Rho kinase belongs to the Rho GTPase family of proteins, which includes the Rho, Rac1 and Cdc42 kinases. One of the best characterized effector molecule of Rho is ROCK, which is a serine/threonine kinase that binds to the GTP-bound form of Rho. The catalytic kinase domain of ROCK, which comprises conserved motifs characteristic of serine/threonine kinases, is found at the N-terminus. ROCK proteins also have a central coiled-coil domain, which includes a Rho-binding domain (RBD). The C-terminus is made up of a pleckstrin-homology (PH) domain with an internal cysteine-rich domain. The coiled-coil domain is thought to interact with other α-helical proteins. The RBD, located within the coiled-coil domain, interacts only with activated Rho GTPases, including RhoA, RhoB, and RhoC. The pH domain is thought to interact with lipid mediators such as arachidonic acid and sphingosylphosphorylcholine, and may play a role in protein localization. Interaction of the pH domain and RBD with the kinase domain results in an auto-inhibitory loop. In addition, the kinase domain is involved in binding to RhoE, which is a negative regulator of ROCK activity.

The ROCK family currently consists of two members, ROCK1 (also known as ROKβ or p160ROCK) and ROCK2 (also known as ROKα). ROCK1 is about 1354 amino acids in length and ROCK2 is about 1388 amino acids in length. The amino acid sequences of human ROCK1 and human ROCK2 are well known. For example, the amino acid sequence of ROCK 1 and ROCK2 can be found at UniProt Knowledgebase (UniProtKB) Accession Number Q13464 and 075116, respectively. The nucleotide sequences of human ROCK1 and ROCK2 can be found at GenBank Accession Number NM_005406.2 and NM_004850, respectively. The nucleotide and amino acid sequences of ROCK1 and ROCK2 proteins from a variety of animals are also well-known and can be found in both the UniProt and GenBank databases.

Although both ROCK isoforms are ubiquitously expressed in tissues, they exhibit differing intensities in some tissues. For example, ROCK2 is more prevalent in brain and skeletal muscle, while ROCK1 is more abundant in liver, testes and kidney. Both isoforms are expressed in vascular smooth muscle and heart. In the resting state, both ROCK1 and ROCK2 are primarily cytosolic, but are translocated to the membrane upon Rho activation. ROCK activity is regulated by several different mechanisms, thus Rho-dependent ROCK activation is highly cell-type dependent, ranging from changes in contractility, cell permeability, migration and proliferation to apoptosis. At least 20 ROCK substrates have been identified. See Hu and Lee, Expert Opin. Ther. Targets 9:715-736 (2005) and Loirand et al, Cir. Res. 98:322-334 (2006) and Riento and Ridley, Nat. Rev. Mol. Cell Biol. 4:446-456 (2003) all of which are incorporated by reference.

The role of ROCK in regulating apoptotic signaling is highly cell-type dependent and stimulus dependent. On the other hand, ROCK has also been associated with mediating cell-survival signals in vitro and in vivo. A ROCK-mediated pro-survival effect has been reported in epithelial cells, cancer cells and endothelial cells, as well as in other cell types. In airway epithelial cells, inhibition with Y-27632 or HA 1077 (also known as fasudil) induces membrane ruffling, loss of actin stress fibers and apoptosis (Moore et al., Am. J. Respir. Cell Mol. Biol. 30:379-387, 2004).

Rho/ROCK activation may also play a pro-survival role during oxidative stress-induced intestinal epithelial cell injury (Song et al., Am. J. Physiol. Cell Physiol. 290:C1469-1476, 2006). ROCK has also been associated with pro-survival events in thyroid cancer cells (Zhong et al., Endocrinology 144:3852-3859, 2003), glioma cells (Rattan et al, J. Neurosci. Res. 83:243-255, 2006), human umbilical vein endothelial cells (Li et al., J. Biol. Chem. 277:15309-15316, 2002), hepatic stelate cells (Ikeda et al., Am. J. Physiol. Gastrointest. Liver Physiol. 285:G880-886, 2003) and human neuroblastoma cells (De Sarno et al., Brain Res. 1041: 112-115, 2005). Evidence of ROCK playing a pro-survival role has also been reported in vivo, for example in vascular smooth muscle cells (Shibata et al, Circulation 103:284-289, 2001) and spinal motor neurons (Kobayashi et al, J. Neurosci. 24:3480-3488, 2004).

As used herein, inhibiting ROCK can mean to reduce the activity, function or expression of at least one of ROCK1 or ROCK2. The activity, function or expression may be completely suppressed, i.e., no activity, function or expression, or the activity, function or expression may simply be lower in treated versus untreated cells. In general, ROCK phosphorylates LIM kinase and myosin light chain (MLC) phosphatase after being activated through binding of GTP-bound Rho. One embodiment of the present invention thus involves blocking the upstream pathway of ROCK1 and/or ROCK2, for example GTP-bound Rho, such that ROCK1 and/or ROCK2 is not activated or its activity is reduced over untreated cells. Other upstream effectors include but are not limited to, integrins, growth factor receptors, including but not limited to, TGF-beta and EGFR, cadherins, G protein coupled receptors and the like. Another embodiment of the present invention thus involves blocking the activity, function or expression of downstream effector molecules of activated ROCK1 and/or ROCK2 such that ROCK1 and/or ROCK2 cannot propagate any signal or can only propagate a reduced signal over untreated cells. Downstream effectors include but are not limited to, Myosin phosphatase-targeting protein (MYPT), vimentin, LIMK, Myosin light chain kinase, NHE1, cofilin, Myosin II and the like. For example, both C3 transferase, a ROCK upstream inhibitor that inhibits the activity of Rho, and blebbistatin, a ROCK downstream inhibitor that inhibits the activity of myosin II, when used in the culture conditions described herein in place of a ROCK inhibitor, affected the cells in such a manner as to allow the cells to bypass differentiation and allow proliferation in vitro. Upstream or downstream inhibition of ROCK, in place of direct ROCK inhibition and in conjunction with the other culture conditions described and required herein, may or may not generate conditionally immortalized islet cells.

The methods of the present invention comprise inhibiting ROCK while culturing the islet cells. In one embodiment, inhibiting ROCK is accomplished by addition of a ROCK inhibitor to the culture medium. In this embodiment where a ROCK inhibitor is added to culture medium.

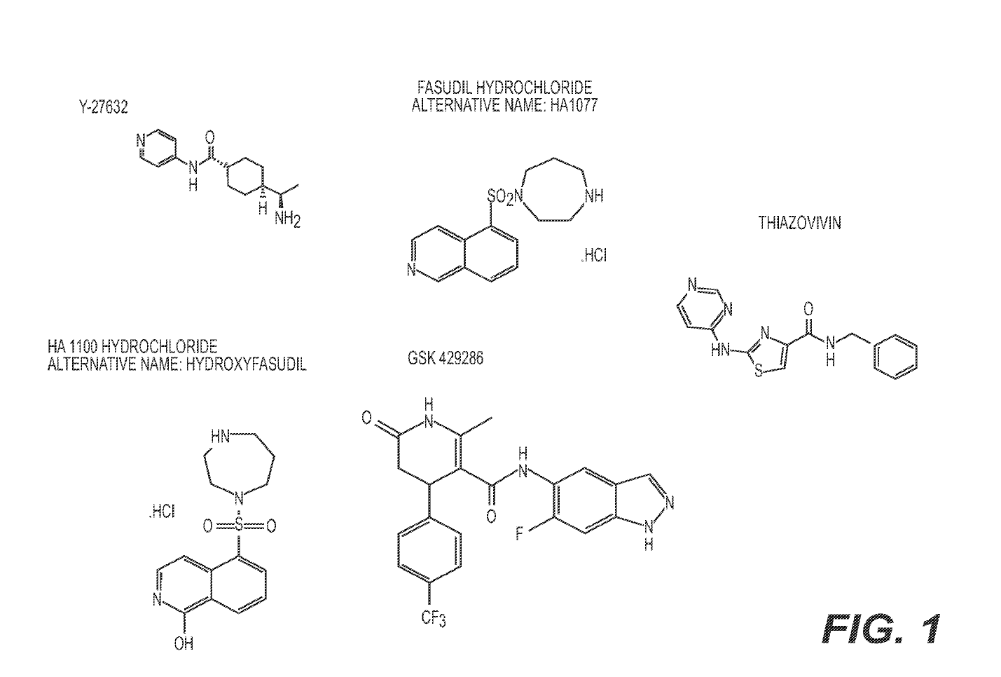

Examples of ROCK inhibitors include but are not limited to Y-27632, HA1100, HA1077, Thiazovivin and GSK429286, the structures of which are depicted in FIG. 1. These compounds are well known and commercially available. Additional small molecule Rho kinase inhibitors include but are not limited to those described in PCT Publication Nos. WO 03/059913, WO 03/064397, WO 05/003101, WO 04/112719, WO 03/062225 and WO 03/062227, and described in U.S. Pat. Nos. 7,217,722 and 7,199,147, and U.S. Patent Application Publication Nos. 2003/0220357, 2006/0241127, 2005/0182040 and 2005/0197328, the contents of all of which are incorporated by reference.

Another way of inhibiting ROCK kinase would be through the use of RNA interference (RNAi). RNAi techniques are well known and rely of double-stranded RNA (dsRNA), where one stand of the dsRNA corresponds to the coding strand of the mRNA that codes for ROCK1, and the other strand is complementary to the first strand. The requirements of optimal RNAi species for a given nucleotide sequence are well-known or can be readily ascertained given the state of the art. For example, it is known that optimal dsRNA is about 20-25 nt in length, with a 2 base overhand on the 3′ end of each strand of the dsRNA, often referred to as short interfering RNAs (siRNA). Of course, other well-known configurations such as short hairpin RNA (shRNA) may also work. shRNAs are one continuous RNA strand where a portion is self-complementary such that the molecule is double-stranded in at least one portion. It is believed that the cell processes shRNA into siRNA. The term RNAi molecule, as used herein, is any double stranded double-stranded RNA (dsRNA), where one stand of the dsRNA corresponds to the coding strand of the mRNA that codes for the target gene to be silenced, and the other strand is complementary to the first strand.

Accordingly, one embodiment of the methods of the present invention involves the use of at least one RNAi molecule and/or at least one antisense molecule, to inhibit the activity of ROCK. In one specific embodiment, the RNAi molecule and/or antisense molecule is specific towards ROCK1. In another embodiment, the RNAi molecule or antisense molecule is specific towards ROCK2. In yet another embodiment, the RNAi molecule and/or antisense molecule is specific towards both ROCK1 and ROCK2. In still another embodiment, at least two RNAi molecules and/or antisense molecules are used, where one is specific towards ROCK1 and the other is specific towards ROCK2.

The RNAi molecules and/or antisense molecules may be part of the cell culture by simply soaking the cells with the naked RNAi molecules and/or antisense molecules as has been reported Clemens, J. C., et al., PNAS, 97(12):6499-6503 (2000), which is incorporated by reference. The RNAi molecules and/or antisense molecules may also be part of a complex, such as a liposomal complex that can be used to insert RNAi molecules or antisense/molecules into the cells.

Liposomes fall into two broad classes. Cationic liposomes are positively charged liposomes which interact with the negatively charged dsRNA molecules to form a stable complex. The positively charged dsRNA/liposome complex binds to the negatively charged cell surface and is internalized in an endosome. Due to the acidic pH within the endosome, the liposomes are ruptured, releasing their contents into the cell cytoplasm (Wang et at., Biochem. Biophys. Res. Commun., 1987, 147, 980-985).

Liposomes that are pH-sensitive or negatively-charged entrap dsRNA rather than complex with it. Since both the dsRNA and the lipid are similarly charged, repulsion rather than complex formation occurs. The dsRNA is thus entrapped in the aqueous interior of these liposomes. pH-sensitive liposomes have been used, for example, to deliver dsRNA encoding the thymidine kinase gene to cell monolayers in culture (Zhou et al., Journal of Controlled Release, 1992, 19, 269-274). One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol. Liposomes that include nucleic acids have been described, for example, in WO 96/40062, U.S. Pat. Nos. 5,264,221, 5,665,710 and Love et al., WO 97/04787 all of which are incorporated by reference.

Another type of liposome, a transfersome, is a highly deformable lipid aggregate which is attractive for drug delivery vehicles. (Cevc et al., 1998, Biochim Biophys Acta. 1368(2): 201-15.) Transfersomes may be described as lipid droplets which are so highly deformable that they can penetrate through pores which are smaller than the droplet. Transfersomes are adaptable to the environment in which they are used, for example, they are shape adaptive, self-repairing, frequently reach their targets without fragmenting, and often self-loading. Transfersomes can be made, for example, by adding surface edge-activators, usually surfactants, to a standard liposomal composition.

Another way ROCK1 and/or ROCK2 RNAi can gain access to the cells in the methods of the present invention is through the use of DNA expression vectors that encode the RNAi molecules and/or antisense molecules. Certain embodiments can utilize only one vector, for example when the RNAi molecule is a shRNA, or when opposing promoters are placed on either side there of the coding sequence for the RNAi molecule. Thus “inhibiting the activity of ROCK” includes the use of DNA that, when transcribed, can block the activity, function or production of ROCK. The liposomal delivery systems described above are one way in which the DNA encoding an RNAi and/or antisense can enter the cell.

Alternatively, the DNA encoding an RNAi and/or antisense can be prepared in a viral vector system that has the capability of entering into cells. These are well-known in the art and include Madzak et al., J. Gen. Virol., 73: 1533-36 (1992) (papovavirus SV40); Berkner et al., Curr. Top. Microbiol. Immunol., 158: 39-61 (1992) (adenovirus); Moss et al., Curr. Top. Microbiol. Immunol., 158: 25-38 (1992) (vaccinia virus); Muzyczka, Curr. Top. Microbiol. Immunol., 158: 97-123 (1992) (adeno-associated virus); Margulskee, Curr. Top. Microbiol. Immunol., 158: 67-93 (1992) (herpes simplex virus (ISV) and Epstein-Barr virus (HBV)); Miller, Curr. Top. Microbiol. Immunol., 158: 1-24 (1992) (retrovirus); Brandyopadhyay et al., Mol. Cell. Biol., 4: 749-754 (1984) (retrovirus); Miller et al., Nature, 357: 455-450 (1992) (retrovirus); Anderson, Science, 256: 808-813 (1992) (retrovirus); C. Hofmann et al., Proc. Natl. Acad. Sci. USA, 1995; 92, pp. 10099-10103 (baculovirus).

In another embodiment, ROCK 1 and/or 2 are inhibited using genetic manipulation techniques, such as, but not limited to, transgenic techniques involving either knockout or dominant negative constructs. Such constructs are disclosed in Khyrul, W., et al., J. Biol. Chem., 279(52):54131-54139 (2004), which is incorporated by reference herein.

As mentioned above, one embodiment of blocking ROCK would be to individually or collectively block or inhibit the upstream or downstream effectors molecules of ROCK using any of the methods described herein, such as but not limited to small molecule inhibitors, RNAi techniques, antisense techniques and/or genetic manipulation. Accordingly, any upstream effectors that could be inhibited include but are not limited to, integrins, growth factor receptors, including but not limited to, TGF-beta and EGFR, cadherins, G protein coupled receptors and the like. In addition, any downstream effectors that could be inhibited include but are not limited to, vimentin, LIMK, Myosin light chain kinase, NHE1, cofilin and the like.

In specific embodiments, the novel compositions of the present invention comprise human serum and at least one ROCK inhibitor in a “base” culture medium such as, but not limited to one or more of Minimal Essential Medium (MEM), DMEM, F12, DMEM-F12, RPMI, Leibovitz's L-15, Glasgow Modified Minimal Essential Medium (GMEM), Iscove's Modified Dulbecco's Medium (IMDM) and Eagle's Minimal Essential Medium (EMEM). In additional specific embodiments, the novel compositions of the present invention comprise insulin, human serum and at least one ROCK inhibitor in a “base” culture medium such as, but not limited to one or more of Minimal Essential Medium (MEM), DMEM, F12, DMEM-F12, RPMI, Leibovitz's L-15, Glasgow Modified Minimal Essential Medium (GMEM), Iscove's Modified Dulbecco's Medium (IMDM) and Eagle's Minimal Essential Medium (EMEM). In additional specific embodiments, the novel compositions of the present invention comprise insulin, hydrocortisone, human serum and at least one ROCK inhibitor in a “base” culture medium such as, but not limited to one or more of Minimal Essential Medium (MEM), DMEM, F12, DMEM-F12, RPMI, Leibovitz's L-15, Glasgow Modified Minimal Essential Medium (GMEM), Iscove's Modified Dulbecco's Medium (IMDM) and Eagle's Minimal Essential Medium (EMEM). In additional specific embodiments, the novel compositions of the present invention comprise insulin, hydrocortisone, cholera toxin, human serum and at least one ROCK inhibitor in a “base” culture medium such as, but not limited to one or more of Minimal Essential Medium (MEM), DMEM, F12, DMEM-F12, RPMI, Leibovitz's L-15, Glasgow Modified Minimal Essential Medium (GMEM), Iscove's Modified Dulbecco's Medium (IMDM) and Eagle's Minimal Essential Medium (EMEM). In additional specific embodiments, the novel compositions of the present invention comprise insulin, hydrocortisone, cholera toxin, epithelial growth factor (EGF), human serum and at least one ROCK inhibitor in a “base” culture medium such as, but not limited to one or more of Minimal Essential Medium (MEM), DMEM, F12, DMEM-F12, RPMI, Leibovitz's L-15, Glasgow Modified Minimal Essential Medium (GMEM), Iscove's Modified Dulbecco's Medium (IMDM) and Eagle's Minimal Essential Medium (EMEM).

In additional embodiments, the novel compositions of the present invention comprise CMRL medium supplemented with L-glutamine, 1% Penicillin/Streptomycin, 10% human serum, Alk5ii inhibitor, T3 and B27, which is a commercially available cell culture supplement. CMRL is a commercially available medium that comprises CaCl₂) (anhydrous), KCl, MgSO4 (anhydrous), NaCl, NaH2PO4.H2O, NaHCO₃, L-Alanine, L-Arginine.HCL, L-Aspartic Acid, L-Cysteine.HCl.H2O, L-Cystine.2HCl, L-Glutamic Acid, Glycine, L-Histidine.HCl.H2O, Hydroxy-L-Proline, L-Isoleucine, L-Leucine, L-Lysine.HCl, L-Methionine, L-Phenylalanine, L-Proline, L-Serine, L-Threonine, L-Tryptophan, L-Tyrosine.2Na.2H2O, Biotin, Folic Acid, Riboflavin, Ascorbic Acid, D-Ca-Pantothenate, Choline Chloride, knositol, Nicotinic Acid, Nicotinamide, PABA, Pyridoxine.HCl, Thiamine.HCl, Thiamine pyrophosphate:Na, Thymidine, 2′-Deoxyadenosine.H2O, 2′-Deoxycytidine.HCl, 2′-Deoxyguanosine.H2O, 5-Methyl-2′-Deoxycytidine, Uridine-5′-triphosphate.3Na.hydrate, Cholesterol, Polysorbate 80, Coenzyme A Li3 Salt.2H2O, b-NAD.hydrate, b-NADP.Na.4H2O, FAD Disodium Salt, Dextrose, Glutathione (reduced), Sodium acetate, Sodium glucuronate.H2O and L-Glutamine.

The range of concentrations of the supplements can vary. For example the range of L-glutamine between about 0.1% to about 20% (vol glutamine/vol CMRL base), 0.5% to about 15%, 1% to about 10% and about 5% to about 10%. The range of serum can vary from between about 0.1% to about 20% (total vol), 0.5% to about 15%, 1% to about 10% and about 5% to about 10%. The range of Alk5i inhibitor can vary from between about 0.01 mM to about 50 mM, from about 0.1 mM to about 40 mM, from about 1 mM to about 30 mm, from about 5 mM to about 25 mM and from about 10 mM to about 20 mM. The range of T3 can vary from between about 0.001 mM to about 50 mM, from about 0.01 mM to about 40 mM, from about 0.1 mM to about 30 mm, from about 0.5 mM to about 25 mM, from about 1 mM to about 20 mM and from about 5 mM to about 10. The range of B27 can vary from between about 0.01% to about 20% (total vol), from 0.1% to about 15%, from 0.5% to about 10% and from about 1% to about 5%. In one specific embodiment, the novel compositions comprise CMRL medium (500 ml) supplemented with L-glutamine (5 ml), 1% Penicillin/Streptomycin (5 ml), 10% human serum (50 ml), Alk5i inhibitor (10 mM at 1000×) and T3 (1 mM at 1000×).

After culturing in the conditions of the present invention, the cells may be removed from these conditions and placed in a cell culture environment where the environment is absent serum and/or absent another component of ICE medium, such as but not limited to a ROCK inhibitor. Any combination of one or two of the components of ICE Medium and the ROCK inhibitor may be absent in the subsequent environment. As used herein, a “subsequent environment” when used in connection with a cell culture environment is a cell culture environment in which at least one of the components of ICE medium is absent. In one embodiment, the ROCK inhibitor is absent in the subsequent environment. In another embodiment, the ROCK inhibitor and serum are absent from the subsequent environment.

In one embodiment, the subsequent environment to the islet cells, the late passage islet cells and/or the conditionally immortalized islet cells is an environment that can promote differentiation (or re-differentiation) and/or does not allow for indefinite proliferation of the islet cells, the late passage islet cells and/or the conditionally immortalized islet cells. The subsequent environment may be an in vivo environment that is similar or identical to a pancreas, i.e., an autologous implant. For example, islet cells that have been cultured according to the methods of the present invention can be reintroduced into the pancreas of the subject from which the islet cells were initially biopsied or isolated. In one specific embodiment, the subsequent environment is a cell culture environment comprising CMRL medium supplemented with T3, Alk5i inhibitor, human serum and, optionally, up to 1% B27 supplement.

The subsequent environment may be an in vitro environment that is that more closely resembles the biochemical or physiological properties of the pancreas once placed in this subsequent environment. The subsequent environment may also be a “synthetic environment” such that factors known to promote differentiation (or re-differentiation) in vitro are added to the cell culture. For example, late passage islet cells, once placed in a subsequent environment that is designed to promote differentiation (or re-differentiation) of the cells, may begin to form grow in a manner and/or express proteins that resemble mature islet cells.

In one embodiment, the islet cells, the late passage islet cells and or the conditionally immortalized islet cells are placed into a subsequent environment that is specific to stimulate differentiation (or re-differentiation) of cells into the islet cells. Such methods of placing the late passage islet cells or conditionally immortalized islet cells in a subsequent environment and promoting or allowing re-differentiation of the cells may be referred to herein as “re-deriving” islet cells. Accordingly, the population of cells that results from the methods of the present invention are termed herein as “re-derived islet cells.” Various environments for culturing epithelial cells are detailed in Culture of Epithelial Cells (Ian Freshney and Mary G. Freshney, Eds. Wiley-Liss, Inc.) (2^{nd} Ed. 2002), which is incorporated by reference.

Alternatively, the cells can be seeded in a subsequent environment into or onto a natural or synthetic three-dimensional cell culture surfaces. One non-limiting example of a three-dimensional surface is a Matrigel®-coated culture surface. Other three dimensional culture environments include surfaces comprising collagen gel and/or a synthetic biopolymeric material in any configuration, such as but not limited to a hydrogel. Of course, a variety of three dimensional culture surfaces may be used simultaneously with the methods the present invention. These three-dimensional cell culture surface environments may or may not promote differentiation (or re-differentiation).

In one embodiment, islet cells, the late passage islet cells and/or the conditionally immortalized islet cells can be genetically modified to express a protein of interest. The genetic modification of the cells would not be a modification designed to immortalize the cells, such as the insertion of a viral protein. Rather, the genetic modification of the cells would be designed to, for example, insert a transgene that codes for a protein. For example, once islet cells are isolated and expanded using the cell culture methods of the present invention, the cells can subsequently be manipulated and a transgene coding for a protein, including but not limited to, a marker protein, can be inserted in the genome of the cells. These cells can then be placed in a subsequent environment, such as an autologous implant into a subject, such that the cells will produce the protein encoded by the transgene.

The methods by which the transgenes are introduced into the cells are standard methods known from the literature for in vitro transfer of DNA into mammalian cells, such as electroporation; calcium phosphate precipitation or methods based on receptor-mediated endocytosis, disclosed in WO 93/07283, which is incorporated by reference. Other methods and materials for inserting a gene of interest into cells are disclosed in Sambrook et al., Molecular Cloning: A Laboratory Manual, Cold Springs Harbor Laboratory Press, Third Edition (2001), which is incorporated by reference.

A wide variety of genes of interest can be expressed in the islet cells, the late passage islet cells and/or the conditionally immortalized islet cells. These genes of interest include, but are not limited to, sequences encoding toxins, enzymes, prodrug converting enzymes, antigens which stimulate or inhibit immune responses, tumor necrosis factors, cytokines, and various proteins with therapeutic applications, e.g., growth hormones and regulatory factors and markers, such as green fluorescent protein and the like.

After transfecting the islet cells, the late passage islet cells and/or the conditionally immortalized islet cells of the present invention, these cells that were successfully transfected can be selected for using markers that are well known in the art. After selection of the successfully transfected cells, the genetically modified islet cells, the late passage islet cells and/or the conditionally immortalized islet cells of the present invention can be cultured using the cell culture techniques of the present invention to produce a population of genetically modified islet cells, late passage islet cells and/or conditionally immortalized islet cells. These cells can subsequently be collected and placed into a subsequent environment as described above, including but not limited to being placed back into the subject, i.e., an autologous implant.

The present invention is also directed to methods of identifying candidate treatments for a subject in need of treatments for which the subject has a condition marked by the presence of abnormal or diseased islet cells. Such conditions marked by the presence of abnormal or diseased islet cells include but are not limited to neoplasias, a hyperplasias or malignant tumors or benign tumors. The methods comprising obtaining a sample of the abnormal islet cells from the subject and culturing the abnormal islet cells according to any of the culture methods of the present invention to produce an in vitro population of abnormal islet cells.

A response profile, as used herein, is a collection of one or more data points that would indicate, e.g., to a clinician, the likelihood that a particular treatment will produce a desired response in the abnormal islet cells if they were in an in vivo setting. A “response” as used in connection with a response profile may or may not be either cell death by any means (necrosis, toxicity, apoptosis etc) or a reduction of the growth rate of the abnormal islet cells. The response profile need not predict a response with 100% accuracy. A response profile can be a single data point or it can be a collection of data.

Any method can be used to identify or determine the response profile of a given population of abnormal islet cells. For example, the response profile may be assessed by sequencing at least part of the DNA or RNA that is isolated from the abnormal islet cells. This may be particularly useful when it is suspected that a virus may be causing the abnormal condition. It is not necessary that all of the DNA/RNA be sequenced to provide at least one data point for the response profile. For example, using well-known techniques involving polymerase chain reaction (PCR), it would currently be a matter of simple procedure to use PCR primers with sequences specific for the DNA/RNA suspected of being present in a PCR reaction to determine if a product is made. If no detectable product is generated after the PCR reaction using specific primers, it may be possible to conclude that the portion of the protein for which the PCR primers are specific may not be present. Likewise, determining the absence of a particular DNA/RNA sequence could also be a data point in a response profile. In this manner, the DNA or RNA is “sequenced” for the purposes of the present invention, although the precise sequence is not determined for the entire DNA/RNA sequence isolated from the cells. Thus, “sequencing” as used herein may or may not result in generating the entire nucleotide sequence of the isolated DNA/RNA. Other methods can also be used to determine the sequence of the isolated DNA/RNA such as, but not limited to Southern blots, Northern blots, RT-PCR, automated sequencing and the like. Methods of sequencing DNA/RNA are well known in the art and need not be repeated herein.

Similarly, the response profile may be assessed by identifying the presence or absence of at least a portion of one mRNA that may be produced in the abnormal islet cells in vitro. Like determining the sequence of the DNA/RNA above, the precise sequence of the mRNA need not be determined for the entire mRNA isolated from the cells. Methods that can also be used to determine the presence or absence of the sequence of the isolated mRNA include but are not limited to Northern blots, RT-PCR, automated sequencing and the like. Methods of identifying the presence or absence of the at least one mRNA are well known in the art and need not be repeated herein.

Similarly, the response profile may be assessed by identifying the presence or absence of at least a portion of one protein that may be produced in the abnormal islet cells in vitro. Like determining the sequence of the DNA/RNA above, the precise amino acid sequence of the present or absent protein need not be determined for the entire protein. Methods that can also be used to determine the presence or absence of the sequence of the isolated protein include but are not limited to Western blots, immunohistochemical methods, ELISA methods, and the like. Methods of identifying the presence or absence of the at least one protein are well known in the art and need not be repeated herein. The presence or absence of a protein, e.g., a receptor, may indicate that the cells are susceptible to a particular treatment that may, for example, result in cell death.

The response profile may be assessed by subjecting the abnormal islet cells in vitro to a chemotherapeutic agent and determining the response of the cells to the chemotherapeutic agent. As used herein, a chemotherapeutic agent is not limited to traditional cancer treatments but is used to indicate a therapeutic treatment of any kind using a chemical entity. In one embodiment, the response to the therapeutic agent can be assessed by determining the therapeutic index of the agent on the cells. Determining the therapeutic index is common in the art and is simply the ratio of the LD₅₀/EC₅₀, with the LD₅₀ representing the median lethal dose and the EC₅₀ representing the half maximal dose of the agent on the cells. Other methods to assess a response to the agent include but are not limited to determining dose response curves, cell survival curves and the like. In one embodiment, the agent that is used to determine the response of the abnormal islet cells to the agent can be the same or a different agent that is later administered to the subject.

The present invention is also directed to methods of identifying an abnormal islet cells in a subject. These methods comprise culturing candidate abnormal islet cells isolated from the subject according to the cell culture methods of the present invention. Once the initial candidate islet cells have been expanded using the methods of the present invention, a cell profile can be determined for the cells to determine if the islet cells are abnormal. At least one feature of the expanded islet cells, the late passage islet cells and or the conditionally immortalized islet cells can be compared to the same feature(s) of normal islet cells. Any difference between abnormal or diseased cells and normal cells can be used, including but not limited to, cell growth characteristics, for example, colony formation on a cell surface, Matrigel™ or other three-dimensional surface. Other means of determining differences between diseased and normal cells include, but are not limited to, assessing the proteomic profile of the cells, assessing the metabolomic profile of the cells, assessing the genomic profile, and/or using other biological assays that will highlight a difference between diseased or abnormal cells and normal cells. A detected difference in the candidate abnormal islet cells and the normal islet cells would indicate that the candidate abnormal islet cells are abnormal compared to normal islet cells.

The present invention is also directed to methods of monitoring the progression of a disease or treatment of a disease in a subject. As used herein, the phrase “monitor the progression” is used to indicate that the abnormal condition in the subject is being periodically checked to determine if an abnormal condition is progressing (worsening), regressing (improving) or remaining static (no detectable change) in the individual by assaying islet cells and/or their cellular contents for various markers of progression or regression. The methods of monitoring may be used in conjunction with other monitoring methods or treatment regimens for an abnormal condition and to monitor the efficacy of these treatments. Thus, “monitor the progression” is also intended to indicate assessing the efficacy of a treatment regimen by periodically assaying islet cells and/or their cellular contents for various markers of progression or regression and correlating any differences in the subject over time with the progression, regression or stasis of the abnormal condition. The methods of monitoring can also be used to determine a suitable follow up therapeutic regimen, after an initial treatment. For example, after an initial treatment islet cells can be biopsied or isolated and the culture methods can be used to generate enough cells in vitro to determine if the genetic makeup or phenotype of the remaining abnormal cells is sufficiently different enough to warrant a new therapy. Thus, in one embodiment, the present invention provides methods of individualizing a therapeutic regimen. Monitoring may also include assessing the levels of a specific marker on the islet cells at two time points from which a sample is taken, or it may include more time points, where any of the levels the marker at one particular time point from a given subject may be compared with the levels of biomarker in the same subject, respectively, at one or more other time points.

The present invention also provides kits for culturing islet cells and/or generating conditionally immortalized islet cells. The kits can include culture vessels, culture media in wet or dry form and/or individual media components such as serum. The kit may or may not include chemicals, such as trypsin, for passaging cells, etc.

### EXAMPLES

### Example 1—Harvesting and Culturing of Primary Islet Cells (HICs)

Human islet cells were isolate at the cGMP facility in the Islet Cell Laboratory at the Georgetown University Hospital according to the methods disclosed in Paget, M., et al., Diabetes Vasc. Disc. Res., 7:4-12 (2007), which is incorporated by reference. Briefly, the cadaveric research donor pancreas was received from WRTC (local OPO). On arrival of the lab, the pancreatic duct was cannulated after trimming. The pancreas was cannulated with a cannula. An enzyme solution containing collagenase HA and Thermolysin (Vitacyte, Indiana, USA) were infused into the pancreas through the cannula. The thoroughly distended pancreas was then digested using the semi-automated method of Ricordi. The digested tissue was recombined. Finally, the human islets were purified using a modified continuous density gradient method with cell processor COBE2991.

The cells were then suspended in DMEM-F12 medium containing 10% human serum (to neutralize the trypsin) and immediately centrifuged to isolate the pelleted cells. Such method of routine isolation and culturing of islet cells are found in Culture of Epithelial Cells (Ian Freshney and Mary G. Freshney, Eds. Wiley-Liss, Inc.) (2^{nd} Ed. 2002), which is incorporated by reference.

After spinning and removal of the supernatant, the pellet was resuspended and plated in ICE medium. Components of the ICE medium included complete DMEM (373 ml) (1×): 500 ml DMEM, 50 ml human serum, 5.5 ml 100× L-glutamine, 5.5 ml 100× Pen/Strep, F12 Nutrient Mix (125 ml), 25 μg/ml Hydrocortisone/0.125 μg/ml EGF Mix (0.5 ml), 5 mg/ml Insulin (0.5 ml), 10 mg/ml Gentamicin (0.5 ml), 11.7 μM Cholera Toxin (4.3 μl), 5 mM Rock Inhibitor (Y-27632) (5 ml). In another embodiment, the pellet was resuspended and cultured as a suspension culture in ICE medium.

The cells were cultured in standard cell culture vessels under normal cells culture conditions, 37° C. at 5% CO₂ and normal atmospheric pressure. Medium was changed every 2-3 days depending upon growth rate.

After the cells reached confluence, the cells were harvested and passaged using standard cell culturing techniques as described in Chapman, S. et al., J. Clin. Invest., 120(7):2619-2626 (2010), which is incorporated by reference.

### Example 2—Cell Markers of Re-Derived Islet Cells

Isolated islets or re-derived islets were fixed in 3% paraformaldehyde for 30 min. The islets were then permeabilized in permeabilization/blocking buffer comprised of phosphate buffered saline (PBS) containing 1% Triton X-100 and 2% filtered bovine serum albumin (blocking agent) at 23° C. for at least 1 hr or overnight at 4° C. Islets were incubated in the same blocking buffer containing the proper dilution of primary antibodies (usually 1:100) for at least 1 hr or overnight at 4° C. Islets were then washed 3× in blocking buffer and then incubated in blocking buffer containing fluorescent secondary antibodies at a concentration of 1:500 to 1:1000 depending on the brand of secondary antibodies, e.g., ALEXA secondary antibodies are linked to strong flour, thus a 1:1000 is a suitable working dilution) for at least 1 hr or overnight at 4° C. Next, islets were washed in PBS containing 1% Triton X 100 three times 30 min each. They were then cultured in DAPI and mounted onto glass slides using a small pipette, covered with a #1 glass cover slip, and visualized using a confocal microscope.

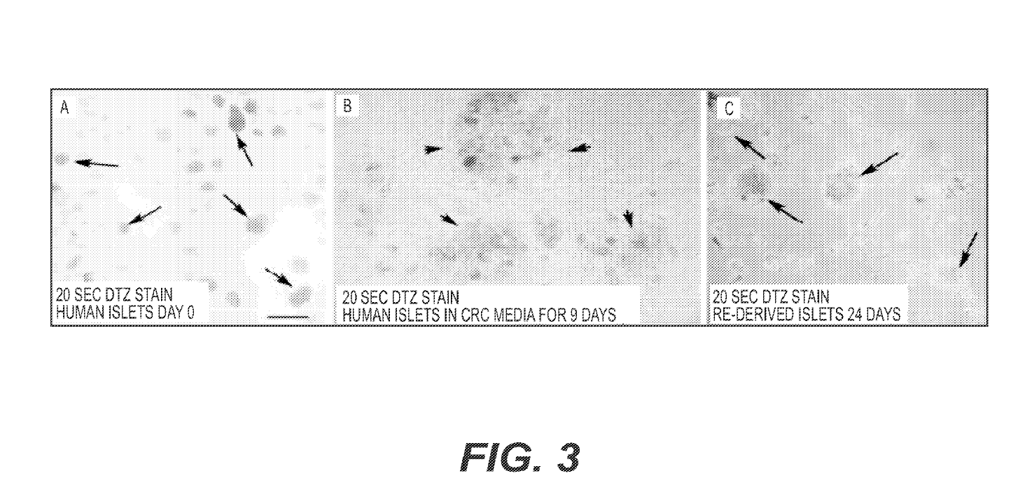

### Example 3—Morphological Architecture of Re-Derived Islet Cells

[FIG. 3 shows representative clusters of islet cells after 9 days in the prescribed culture condition. The isolated primary islets were stained using a 20-second stain in dithizone (DTZ), followed by culture in the culture conditions described herein (ICE Medium). FIG. 3A shows islets shortly after isolation, which stained positively for DTZ. FIG. 3B shows the same cells after nine days in the culture conditions described herein (ICE Medium). The islets re-enter the cell cycle, enabling indefinite passaging. Arrows point to small clusters of cells. No cells, however, stained for DTZ. FIG. 3C shows the cells 14 days in standard CMRL medium used for islet cell culture. The islet clusters stain positive for DTZ (arrows). Scale bar in A=100 μm for A and 50 μm for B and C.

### Example 4—Comparison of Response to Glucose for Re-Derived Islets and Normal Islets

A general ELISA technique was performed for measuring insulin or C-Peptide levels released by islets. In brief, primary islets, or re-derived islets were subjected to either 1 mm glucose or 10 mm glucose in Hank's Balanced Salt Solution (HBSS from Thermo Fisher Scientific Inc.) for 30 min. The HBSS is collected and 10-20 microliters is subjected to any general insulin or C-peptide detection 96 well ELISA kit. Following the kit directions, the ELISA plate is read on a general plate reader. The results were plotted on the standard curve also provided within the ELISA kit

### Example 5—Identification of Candidate Therapeutic Agents for an Individual

A needle biopsy from a subject is obtained is processed according to the methods described herein to generate conditionally immortalized islet cells. mRNA is extracted from the cells and specific primers and rt-PCR is used evaluate gene expression of markers for diseased cells, such as insulin, somatostatin, glucagon, polypeptide (PP) and ghrelin or lack thereof of any of these markers. Based on this genetic analysis, an alternative pathway to treat the subject can be determined.

### Example 6—Establishment of a Cell Line from a Single Needle Biopsy

In a separate experiment, a needle biopsy specimen from a rat pancreas is used in the cell culture methods of the present invention. The rat pancreatic islet cells will proliferate well and a cell line can be established that can be used in vitro studies. Thus, a sufficient number of islet cells to generate the cell lines is obtained in a single needle biopsy. This will greatly expand the capability for performing genetic, biochemical and molecular studies on very small clinical samples of islet cells.

### Example 7—Glucose Challenge of Re-Derived Islet Cells

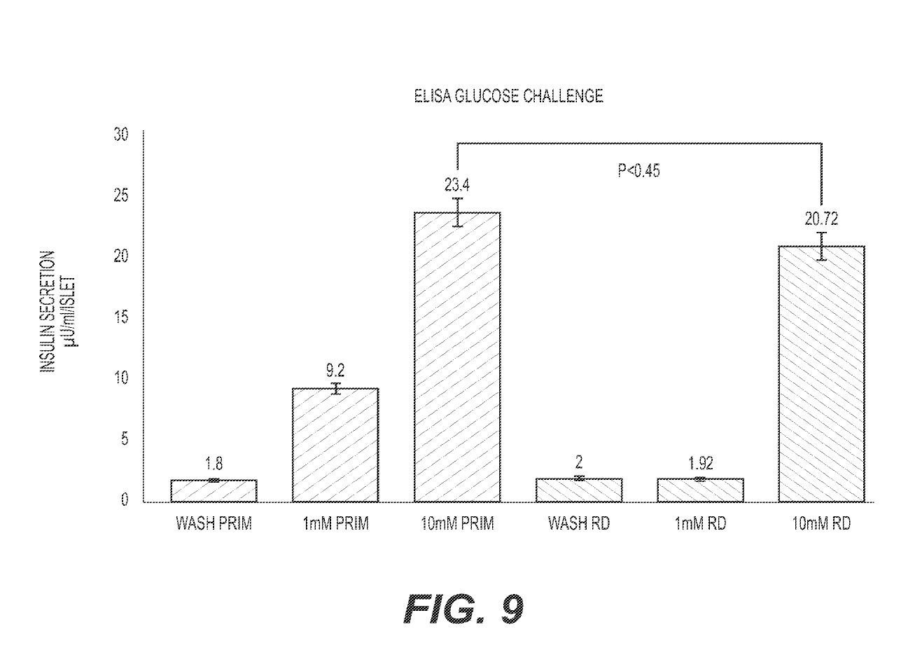

FIG. 9 shows insulin secretion from single islets after being challenged with 1 mM glucose and then 10 mM glucose. The wash was used as a control to show that islets were not secreting insulin prior to glucose challenge. After three expansions in ICE medium and then culturing in medium containing growth factors to induce re-derivation back into islets, the re-derived (RD) islets secrete virtually the same amount of insulin in response to 10 mM glucose when compared to control primary islets freshly isolated from a healthy pancreas. There was no significant difference between the 10 mM glucose stimulated primary islets and 10 mM glucose stimulated RD-islets.

The results here were obtained by performing a 1 mM glucose challenge for 45 minutes, followed by a 10 mM glucose challenge for 45 minutes on single islets. The glucose-challenged cultures were then subjected to ELISA (Millipore, Inc.) and measured in micro Units/ml of insulin, based on a standard curve generated using the ELISA kit instructions. The data were then combined to acquire the insulin secretion average and the standard deviation, which were then used to generate a student t-test to determine statistical significance.

The Examples of Embodiments disclosed herein are meant to be illustrative and are not intended to limit the scope of the present invention in any manner.

What is claimed is:

1. A method of continuously culturing pancreatic islet cells, the method comprising
a) culturing the cells in the presence of a cell culture medium, and
b) inhibiting the activity of Rho kinase (ROCK) during culturing.

2. The method of claim 1, wherein the pancreatic islet cells are primary cells.

3. The method of claim 1, wherein the pancreatic islet cells are not primary cells.

4. The method of claim 1, wherein the pancreatic islet cells are tumor cells.

5. The method of claim 1, wherein the cell culture medium comprises serum or a serum replacement.

6. The method of claim 5, wherein the serum is human serum.

7. The method of claim 1, wherein the ROCK is Rho kinase inhibitor 1 (ROCK 1), Rho kinase inhibitor 2 (ROCK 2) or both.

8. The method of claim 1, wherein inhibiting the activity of ROCK comprises culturing the pancreatic islet in the presence of a small molecule ROCK inhibitor.

9. The method of claim 8, wherein the small molecule ROCK inhibitor is selected from the group consisting of Y-27632, HA1100 hydrochloride, HA1077 and GSK429286.

10. The method of claim 8, wherein inhibiting the activity of ROCK comprises culturing the pancreatic islet cells in the presence of an RNA interference (RNAi) molecule specific for ROCK 1, ROCK 2 or both.

11. The method of claim 1, further comprising
c) passaging the pancreatic islet cells after inhibiting ROCK, and
d) placing the passaged cells in cell culture environment in which ROCK is not being inhibited.

12. The method of claim 11, wherein the environment in which ROCK is not being inhibited is a three-dimensional cell culture environment.

13. A population of conditionally immortalized pancreatic islet cells.

14. The cell population of claim 13, wherein the conditionally immortalized pancreatic islet are derived from normal cells.

15. The cell population of claim 13, wherein the conditionally immortalized pancreatic islet cells are derived from tumors.

16. A method of stimulating growth of pancreatic islet cells, the method comprising
a) culturing the cells in the presence of a cell culture medium, and
b) inhibiting the activity of Rho kinase (ROCK) during culturing,
whereby culturing the pancreatic islet cells while inhibiting the activity of the Rho kinase will stimulate the growth of the pancreatic islet cells.

17. The method of claim 16, wherein the pancreatic islet cells are primary cells.

18. The method of claim 16, wherein the pancreatic islet cells are not primary cells.

19. The method of claim 16, wherein the pancreatic islet cells are tumor cells.

20. The method of claim 16, wherein the cell culture medium comprises serum or a serum replacement.

21. The method of claim 20, wherein the serum is human serum.

22. The method of claim 16, wherein the ROCK is Rho kinase inhibitor 1 (ROCK 1), Rho kinase inhibitor 2 (ROCK 2) or both.

23. The method of claim 16, wherein inhibiting the activity of ROCK comprises culturing the pancreatic islet cells in the presence of a small molecule ROCK inhibitor.

24. The method of claim 23, wherein the small molecule ROCK inhibitor is selected from the group consisting of Y-27632, HA1100 hydrochloride, HA1077 and GSK429286.

25. The method of claim 16, wherein inhibiting the activity of ROCK comprises culturing the pancreatic islet cells in the presence of an RNA interference (RNAi) molecule specific for ROCK 1, ROCK 2 or both.

26. A method of identifying a candidate treatment for a subject in need of treatment of a condition that is marked by the presence of abnormal pancreatic islet cells, the method comprising
a) obtaining a sample of the abnormal pancreatic islet cells from the subject,
b) culturing the abnormal pancreatic islet cells in the presence of a cell culture medium and at least one Rho kinase (ROCK) inhibitor, to produce a population of abnormal pancreatic islet cells in vitro,
c) determining a response profile of at least a portion of the abnormal pancreatic islet cells in vitro, and
d) identifying a candidate treatment for the subject based on the determined response profile.

27. The method of claim 26, wherein the response profile is at least partially determined by identifying the sequence of at least one portion of DNA extracted from the abnormal pancreatic islet cells in vitro.

28. The method of claim 26, wherein the response profile is at least partially determined by identifying at least one mRNA that is produced in the abnormal pancreatic islet cells in vitro.

29. The method of claim 26, wherein the response profile is at least partially determined by identifying at least one mRNA that is not produced in the abnormal pancreatic islet cells in vitro.

30. The method of claim 26, wherein the response profile is at least partially determined by identifying one or more proteins that the abnormal pancreatic islet cells in vitro express.

31. The method of claim 26, wherein the response profile is at least partially determined by identifying one or more proteins that the abnormal pancreatic islet cells in vitro do not express.

32. The method of claim 26, wherein the response profile is at least partially determined by subjecting the abnormal pancreatic islet cells in vitro to a therapeutic agent and determining the therapeutic index of the therapeutic agent on the abnormal pancreatic islet cells in vitro.

33. A method of identifying an abnormal pancreatic islet cell in a subject, the method comprising
a) culturing at least one candidate abnormal pancreatic islet cell isolated from the subject in the presence a cell culture medium and at least one Rho kinase (ROCK) inhibitor, to produce a population of candidate abnormal pancreatic islet cells in vitro,
b) determining a profile of at least a portion of the population of candidate abnormal pancreatic islet cells in vitro, and
c) comparing at least one feature of the candidate abnormal pancreatic islet cells to the same feature of normal pancreatic islet cells to determine if there is a difference between the candidate abnormal pancreatic islet cells and the normal pancreatic islet cells,
wherein a difference indicates that the candidate abnormal pancreatic islet cells are abnormal compared to normal pancreatic islet cells.

34. The method of claim 33, wherein the profile is at least partially determined by identifying at least one mRNA that is produced in the candidate abnormal pancreatic islet cells in vitro.

35. The method of claim 33, wherein the profile is at least partially determined by identifying at least one mRNA that is not produced in the candidate abnormal pancreatic islet cells in vitro.

36. The method of claim 33, wherein the profile is at least partially determined by identifying one or more proteins that the candidate abnormal pancreatic islet cells in vitro express.

37. The method of claim 33, wherein the profile is at least partially determined by identifying one or more proteins that the candidate abnormal pancreatic islet cells in vitro do not express.

38. The method of claim 33, wherein the profile is at least partially determined histologically.

39. A method of administering autologous pancreatic islet cells to a subject in need of additional pancreatic islet cells, the method comprising
a) obtaining a sample of pancreatic islet cells from the subject,
b) culturing the pancreatic islet cells in the presence of a cell culture medium and at least one Rho kinase (ROCK) inhibitor, to produce a population of autologous pancreatic islet cells in vitro,
c) collecting the population of autologous pancreatic islet cells in vitro, and
d) administering the collection of autologous pancreatic islet cells to the subject in need of autologous pancreatic islet cells.

40. A method of administering autologous, genetically modified pancreatic islet cells to a subject in need of additional pancreatic islet cells, the method comprising
a) obtaining a sample of pancreatic islet cells from the subject,
b) culturing the pancreatic islet cells in the presence of a cell culture medium and at least one Rho kinase (ROCK) inhibitor, to produce a population of pancreatic islet cells in vitro,
c) genetically modifying at least a portion of the population of pancreatic islet cells in vitro,
d) selecting for the pancreatic islet cells that were genetically modified,
e) culturing the selected genetically modified pancreatic islet cells in the presence of a cell culture medium and at least one Rho kinase (ROCK) inhibitor, to produce a population of autologous genetically modified pancreatic islet cells in vitro,
f) collecting the population of autologous, genetically modified pancreatic islet cells in vitro, and
g) administering the collection of autologous, genetically modified pancreatic islet cells to the subject in need of additional pancreatic islet cells.

41. A composition comprising human serum human serum, hydrocortisone, epithelial growth factor (EGF), insulin, cholera toxin and at least one Rock Inhibitor.

42. The composition of claim 41, wherein the composition further comprises amino acids and vitamins.

43. The composition of claim 42, wherein the composition further comprises glucose.

44. A composition comprising human serum human serum, T3, Alk5i inhibitor, glutathione, dextrose, thymidine and cholesterol.

45. The composition of claim 44, wherein the composition further comprises amino acids and vitamins.

46. The composition of claim 45, wherein the composition further comprises glutamine.
